(19) United States
(12) Plant Patent Application Publication
(10) Pub. No.: US 2003/0192098 P1
(43) Pub. Date: Oct. 9, 2003

(54) Chrysanthemum plant named 'Chamidi'

(76) Inventor: Jean-Pierre Challet, Nuaille (FR)

Correspondence Address: BURNS DOANE SWECKER & MATHIS L L P, POST OFFICE BOX 1404, ALEXANDRIA, VA, 22313-1404, US

(21) Appl. No.: 10/114,446

(22) Filed: Apr. 3, 2002

(51) Int. Cl. A01H 5/00

(52) U.S. Cl. PLT/289

(57) ABSTRACT

A new and distinct Chrysanthemum cultivar is provided that is a mutation of the ‘Chanizet’ variety (U.S. Plant patent application No. ______, filed concurrently herewith). Attractive ball-shaped bright yellow blossoms are formed in profusion. Such blossoms commonly are approximately 12 to 13 cm in diameter when fully open. The growth habit is low and compact and well-branched and yields a generally uniform and spherical overall plant. The foliage is glossy dark green and contrasts well with the bright yellow blossom coloration, and the stems are extremely stiff. The plant grows well in pots and is well suited for use as an attractive potted gift plant.

### BOTANICAL/COMMERCIAL CLASSIFICATION

Dendranthema grandifloria/decorative Pot Mum

### VARIETAL DENOMINATION

cv. ‘Chamidi’

### SUMMARY OF THE INVENTION

The present invention comprises a new and distinct cultivar of Chrysanthemum, botanically known as Dendranthema grandiflora, and hereafter is referred to by the cultivar name ‘Chamidi’.

The new cultivar of the present invention was created at Nuaillé, France when plants of the ‘Chanizet’ cultivar (U.S. Plant patent application No. ______, filed concurrently herewith) were irradiated with gamma radiation. Cuttings of the irradiated plants were used to produce new plants that were carefully studied. The new cultivar of the present invention was selected from among the rooted cuttings at Nuaillé, France, primarily because of its distinctive and attractive flower coloration.

It was found that the new Chrysanthemum cultivar of the present invention displays:

(a) a low compact, well-branched, and generally spherical growth habit,

(b) forms attractive ball-shaped long-lasting decorative uniform bright yellow blossoms,

(c) forms extremely stiff stems,

(d) forms attractive dark green leaves having shiny upper surfaces, and

(e) an ability to grow well in pots to provide an attractive potted gift plant.

The new cultivar can be grown singly or in clumps in pots. It also can be grown in the landscape. The bright yellow blossoms contract nicely with the shiny dark green foliage. The blossoms have been found to be long lasting and commonly last up to four weeks on the plant. Pinching is helpful to further enhance branching; however, such pinching is not necessary since the plant already is naturally self-branching.

The new cultivar can be readily distinguished from its ‘Chanizet’ parent in view of the different blossom coloration. The blossoms of the cultivar of the present invention are a uniform bright yellow while those of the ‘Chanizet’ cultivar are bronze in coloration.

Asexual reproduction of the new cultivar by cuttings as performed at Nuaillé, France, in a controlled environment has demonstrated that the characteristics of the new cultivar are firmly fixed and are retained through successive generations of asexual propagation.

‘Chamidi’ has not been observed under all possible environmental conditions to date. Accordingly, it is possible that the phenotype may vary somewhat with variations in the environment, such as temperature, light, day length, contact with pesticides and/or subjection to grown regulation treatments.

### BRIEF DESCRIPTION OF THE PHOTOGRAPH

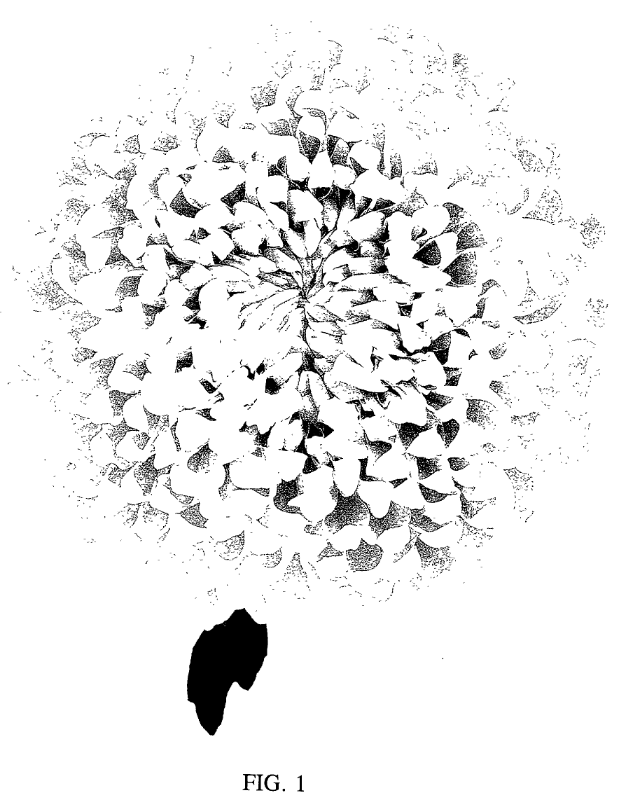

The accompanying FIG. 1 depicts a typical blossom and foliage of the new ‘Chamidi’ cultivar while growing in a pot in a greenhouse at Nuaillé, France. The attractive ball-shaped bright yellow blossom configuration is illustrated.

### DETAILED DESCRIPTION

The plants described were grown in pots in a greenhouse at Nuaillé, France. No growth regulation was used. The growing conditions approximate those commonly utilized for the commercial production of decorative pot mums.

General Appearance:

Short and stocky with a generally spherical growth habit.

Foliage:

Attractive dark green. The upper surfaces of the leaves are shiny and tend to glisten in the sunlight.

Stems:

Extremely stiff.

Flowers:

Possess a decorative uniform bright yellow coloration. The configuration is generally ball-shaped and the diameter is approximately 12 to 13 cm when fully expanded.

Flower Duration:

Very long lasting with the blossoms commonly lasting up to approximately four weeks on the plant.

Response Time:

The time between the beginning of the short day period and the flowering date at normal temperature and light intensity is approximately ten weeks.

Usage:

Decorative pot mum.

I claim:

1. A new and distinct cultivar of Chrysanthemum plant that displays:
(a) a low compact, well-branched, and generally spherical growth habit,
(b) forms attractive long-lasting decorative ball-shaped uniform bright yellow blossoms,
(c) forms extremely stiff stems,
(d) forms attractive dark green leaves having shiny upper surfaces, and
(e) an ability to grow well in pots to provide an attractive potted gift plant;
substantially as illustrated and described.
